(19) United States
(12) Patent Application Publication
(10) Pub. No.: US 2003/0118640 A1
(43) Pub. Date: Jun. 26, 2003

(54) Pharmaceutical formulations comprising sodium amoxycillin and potassium clavulanate

(76) Inventor: Malcolm Dash, West Sussex (ES)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/168,200

(22) PCT Filed: Dec. 19, 2000
(86) PCT No.: PCT/GB00/04911

(30) Foreign Application Priority Data
Dec. 23, 1999 (GB) 9930578.1

(51) Int. Cl. A61K 31/43; A61K 31/407; A61K 9/20; A61K 9/36; A61K 9/32

(52) U.S. Cl. 424/465; 514/210.09; 514/192; 424/480; 424/482

(57) ABSTRACT

A tablet formulation comprising amoxycillin and potassium clavulanate, in a weight ratio amoxycillin:clavulanate between 1:1 to 20:1 (expressed as the weight of the corresponding parent acids) inclusive, wherein the amoxycillin is sodium amoxycillin or a mixture of sodium amoxycillin and amoxycillin trihydrate and the tablet has an enteric film coating is of use in treating bacterial infection.

The present invention relates to medicaments for oral administration in the treatment of bacterial infections, comprising amoxycillin and salts of clavulanic acid.

Amoxycillin and its derivatives, e.g. amoxycillin trihydrate, are known (e.g. GB 1241844) as antibacterial agents useful in the treatment of gram-negative and gram-positive bacterial infections. Clavulanic acid and its derivatives, e.g. its salts such as potassium clavulanate, are known (e.g. GB 1508977) as β-lactamase inhibitors which inhibit the activity of β-lactamase enzymes produced by bacteria and which confer antibiotic resistance by destroying β-lactam antibiotics such as amoxycillin. The terms “amoxycillin” and “clavulanate” used herein unless otherwise specified include both the free parent acids and derivatives such as salts thereof. The use of clavulanate in combination with amoxycillin consequently enhances the effectiveness of amoxycillin.

Amoxycillin is available in a variety of forms, for instance, amoxycillin trihydrate, anhydrous amoxycillin and alkali metal salts of amoxycillin such as sodium amoxycillin. Amoxycillin trihydrate is generally preferred for tablet formulations on account of its favourable compression properties. The sodium salt however has superior solubility and is used in injectable formulations.

GB 2 005 538-A (Beecham Group) describes tablet formulations of potassium clavulanate in combination with amoxycillin trihydrate within the ratios amoxycillin:clavulanic acid 1:1 to 6:1, (expressed in terms of the weight of parent compound amoxycillin or clavulanic acid, this terminology being used throughout this description unless otherwise stated). WO 95/28927 (SmithKline Beecham) describes tablets comprising a compacted mixture of 750-950 mg amoxycillin and a corresponding amount of clavulanate such that the ratio of amoxycillin to clavulanate is between 6:1 and 8:1. Further tablet formulations are described in EP 0 049 061-A (Beecham Group) and WO 92/19227 (SmithKline Beecham), the tablets being optionally coated with an enteric coating such as cellulose acetate phthalate. This dissolves at pH 6.5. WO 95/25516 (SmithKline Beecham) describes formulations comprising spherical granules of amoxycillin optionally with clavulanate which may be coated with an enteric coating. WO 95/28148 (SmithKline Beecham) describes tablet formulations comprising a core containing clavulanate coated with a release retarding coating which may be an enteric polymer. No enteric coated tablet formulations have however been developed for commercial use.

WO 98/22091 (Yissum Research Development Company of the Hebrew University of Jerusalem) describes a controlled release formulation of amoxycillin in which the amoxycillin component may be provided as a mixture of amoxycillin trihydrate and sodium amoxycillin.

WO 98/40054 (Astra Aktielbolag) describes an enteric coated oral dosage form comprising sodium amoxicillin, developed for use in the treatment of H pylori infections.

WO 00/61115 and 00/61116 (SmithKline Beecham, published after the priority date of the present application) describe modified release formulations of amoxycillin and amoxycillin/clavulanate in which the amoxycillin component may be provided as a mixture of amoxycillin trihydrate and sodium amoxycillin, in a ratio 3:1 to 1:3, preferably 2:1 to 2:3; more preferably 3:2 to 1:1.

Gastric intolerance, manifested in symptoms such as loose stools, is perceived in some countries to be a side effect associated with the use of amoxycillin/potassium clavulanate. Accordingly, any measures, such as revised formulations, which can mitigate this would be advantageous. An enteric film coated tablet formulation may be of use in this context.

Use of an enteric film coated formulation may however result in the modification of the pharmacokinetic profile compared with the conventional, immediate release, formulation, particularly for a tablet comprising a large amount of amoxycillin, present as the relatively insoluble amoxycillin trihydrate. There thus remains the need to develop enteric formulations in which the pharmacokinetic profile is not adversely affected. It has been found that this may be achieved by using a more soluble form of amoxycillin.

Accordingly the present invention provides a tablet formulation comprising amoxycillin and potassium clavulanate, in a weight ratio amoxycillin:clavulanate between 1:1 to 20:1 (expressed as the weight of the corresponding parent acids) inclusive, wherein the amoxycillin is sodium amoxycillin or a mixture of sodium amoxycillin and amoxycillin trihydrate and the tablet formulation has an enteric film coating.

The use of an enteric coating delays dissolution of the actives in the tablet core, thereby protecting these from the acidic environment of the stomach. The use of sodium amoxycillin ensures that once the enteric coat is dissolved, as the pH increases, the amoxycillin will be made rapidly available, as sodium amoxycillin is more soluble than other forms.

The sodium amoxycillin may be in the spray dried (GB 1576731, Beecham Group) or crystallised (EP 0 131 147-A, Beecham Group) forms.

It is preferred to use a combination of sodium amoxycillin and amoxycillin trihydrate. The presence of amoxycillin as the trihydrate improves the compressibility of the formulation, to the extent that further excipients to enhance compressibility may be omitted.

Preferably, the ratio of sodium amoxycillin to amoxycillin trihydrate is in the ratio between 2:1 and 10:1, more preferably 3:1 to 5:1, most preferably about 4:1 (expressed as the weight of the corresponding parent acid).

Typical ratios of amoxycillin: clavulanate include 2:1, 4:1, 7:1, 8:1, 14:1 and 16:1.

Preferably, clavulanate is used in the form of the potassium salt.

As used herein, the term ‘enteric film coating’ refers to a film coating which is preferentially soluble in the less acid environment of the intestine relative to the more acid environment of the stomach, thereby allowing the medicament to pass through the stomach and into the small intestine from where it is absorbed. Suitably, the coating dissolves at a pH of about at least 5.5.

An enteric film coating may be an essentially conventional film coating material, for example, an enteric polymer such as cellulose acetate phthalate, polyvinylacetate phthalate, polyvinylbutyrate acetate, vinyl acetate-maleic anhydride copolymer, styrene-maleic mono-ester copolymer, methacrylic acid copolymers, methacrylate-methacrylic acid-octyl acrylate copolymer, etc. These may be used either alone or in combination, or together with other polymers than those mentioned above. The enteric film coating may also include insoluble substances which are neither decomposed nor solubilized in living bodies, such as alkyl cellulose derivatives such as ethyl cellulose, cross-linked polymers such as styrene-divinylbenzene copolymer, polysaccharides having hydroxyl groups such as dextran, cellulose derivatives which are treated with bifunctional cross-linking agents such as epichlorohydrin, dichlorohydrin, 1,2-, 3,4-diepoxybutane, etc. The enteric film coating may also include starch and/or dextrin.

Preferred enteric polymers include pharmaceutically acceptable methacrylic acid copolymers (polymethacrylates) of methacrylic acid and an acrylic or methacrylic ester such as those described in the USP/NF, and such polymers of types A, B and C as described therein may be suitable. Suitable such methacrylic acid copolymers are anionic in character and based on methacrylic acid and methyl or ethyl methacrylate, for example having a ratio of free carboxyl groups: esterified carboxyl groups of 1:>3, e.g. around 1:1 or 1:2, and with a mean molecular weight greater than 100,000, typically about 135,000.

Suitable such copolymers are available under the trade name Eudragit™, for instance:

the Eudragit L series in which the ratio of free carboxyl groups to the ester is approximately 1:1, e.g. Eudragit L 12.5™, Eudragit L 12.5P™, Eudragit L100™, Eudragit L 100-55™, Eudragit L-30™ and Eudragit L-30 D-55™; and

the Eudragit S™ series in which the ratio of free carboxyl groups to the ester is approximately 1:2, e.g. Eudragit S 12.5, Eudragit S 12.5P™, Eudragit S100™.

Preferably, the enteric film coating is a fully polymerised copolymer of methacrylic acid and ethyl acrylate, for instance poly(methacrylic acid, ethyl acrylate) 1:1 which is provided as an aqueous dispersion in the product Eudragit L 30 D-55 which corresponds to USP/NF methacrylic acid copolymer, type C and as a white free flowing powder in the product Eudragit L 100-55.

A further suitable film coating comprises polyvinyl acetate phthalate and is available under the trade name Opadry OY-A-7308 from Colorcon Ltd, Cray Orpington, Kent, England.

The above methacrylic acid copolymers and polyvinyl acetate phthalate are enteric polymers, for example having a solubility in aqueous media at pH 5.5 and above.

The above methacrylic acid copolymers and polyvinyl acetate phthalate may be used either alone or with a plasticiser. The choice of plasticiser will depend upon whether an aqueous or non-aqueous medium is used, for example suitable plasticisers for an aqueous medium include propylene glycol, triethyl citrate or acetyl triethyl citrate, and for a non-aqueous medium include these and also dibutyl or diethyl phthalate. The enteric film coating may also include an anti-tack agent such as talc, silica or glyceryl monostearate. The quantity of plasticiser and anti-tack agent may be generally conventional to the art. Typically the coating may include around 10-25 wt. % plasticiser and around 5-20 wt. % of anti-tack agent.

An enteric film coating may be applied to the core by dissolving or suspending the enteric coating materials in a suitable medium, such as water, methanol, ethanol, iso-propanol, acetone, methyl ethyl ketone, methylene chloride, ethylene chloride, ethyl acetate, etc. or mixtures thereof, and the resultant solution or suspension may be sprayed on the core to coat them, followed by drying sufficiently with an air flow and screening.

In the case of the preferred enteric film coating materials referred to above, the enteric film coating material may be dissolved or suspended in a solvent, for example water, and coated onto the core using a perforated coating pan. If water is used, preferably an anti-foaming agent, such as activated polymethylsiloxane, is also included.

It may be desirable to first apply one or more sub-coats to the core, before application of the enteric coating layer, the sub-coat consequently lying beneath the enteric coating. Suitable sub-coat materials include hydroxypropylmethyl cellulose. It may also be desirable to apply one or more over-coats after application of the enteric coating layer, the over-coat consequently lying over the release retarding coating. Suitable over-coat materials include hydroxypropylmethyl cellulose. The over-coat may be of the same material as the sub-coat. Typically such coatings may be applied by known techniques of aqueous film coating.

Tablets of the invention may suitably contain 50 wt. % or more, for example around 65-75 wt. % of the combination of amoxycillin (sodium salt plus trihydrate, if present) and clavulanate, e.g. typically 70 wt. %±2 wt %.

Suitably the film coating is applied so as to deposit a weight of dried film materials corresponding to around 1.0-10.0 wt. % of the total coated tablet weight, suitably about 5%.

Tablet formulations of the invention may also include one or more other additional excipients etc. conventionally used in tablets. For example, tablet formulations may contain one or more conventional diluents such as microcrystalline cellulose (which can also function as a compression aid) e.g. comprising around 20-35 wt % of the tablet e.g. 25-30 wt %; disintegrants such as sodium starch glycolate or crospovidone, e.g. comprising 0.5-3.5 wt % of the tablet e.g. 1.75-2.25 wt %; lubricants such as magnesium stearate e.g. comprising 0.5-1.5 wt % of the tablet e.g. 0.75-1.25 wt % and glidants, such as colloidal silicon dioxide, e.g. comprising 0.25-1.0 wt % of the tablet e.g. 0.5-0.9 wt %. Although the above-listed classes and examples of excipients, together with the active ingredients may make up the 100% uncoated core weight of the tablet, in addition the tablet forms may contain colourants, desiccants etc. conventional to the dosage form in question up to the 100% uncoated core weight of the tablet.

Tablets of the invention may be made by conventional tablet manufacturing techniques, e.g. blending of the ingredients followed by dry compaction, granulation then compaction of the granulate to form the compacted tablet core. A suitable granulate may be produced for example by slugging or roller compaction. The use of roller compaction to prepare granules comprising amoxycillin and potassium clavulanate is described in WO 92/19227 and WO 95/28927 (both to SmithKline Beecham).

The tablets of the present invention may be provided as monolith tablets, of substantially uniform composition. Alternatively, tablets of the present invention may be provided as bilayer tablets in which the amoxycillin trihydrate and sodium amoxycillin components are provided as separate layers with the potassium clavulanate in either or both layers, by analogy with the modified release bilayer tablets described in WO 00/6116 (SmithKline Beecham).

Potassium clavulanate is known to be highly sensitive to moisture so that it is preferred that the preparation of the formulations of the invention is carried out under conditions of low humidity, e.g. less than 30% RH, more suitably less than 20% RH. ideally as low as possible.

Preferably the tablets are packaged in a container that inhibits the ingress of atmospheric moisture, e.g. blister packs or tightly closeable bottles etc. as conventional in the art. Preferably bottles also include a desiccant material to preserve the clavulanate.

Suitably, tablets according to the present invention are provided in convenient dosage amounts, reflecting the dosage amounts already available, for instance tablets comprising nominally 125/62.5, 250/62.5, 250/125, 500/62.5, 500/125, 875/125, 1000/125 and 1000/62.5 mg amoxycillin/clavulanate.

It will be appreciated that the principle hereinbefore described is also applicable to tablet formulations comprising, amoxycillin alone and no clavulanate. The present invention includes such tablet formulations comprising amoxycillin alone and no clavulanate.

Tablets of this invention may be provided for treatment of bacterial infections generally, for example one or more of inter alia upper respiratory tract infections, lower respiratory tract infections, genito- urinary tract infections and skin and soft tissue infections.

The invention will now be described by way of example only.

### EXAMPLE 1

### 875/125 Tablet with an Eudragit Enteric Polymer Coating

TABLE 1
Formula for Augmentin Core.

| Ingredients | mg/Tablet |
|---|---|
| Sodium Amoxycillin (equivalent to | 700.0 |
| amoxycillin fa) | |
| Amoxycillin Trihydrate (equivalent to | 175.0 |
| amoxycillin fa) | |
| Potassium Clavulanate (equivalent to | 125.0 |
| clavulanic acid fa) | |
| Sodium Starch Glycolate | 29.0 |
| Colloid Silicon Dioxide | 10.0 |
| Magnesium Stearate | 14.5 |
| Microcrystalline Cellulose to | 1450.0 |

TABLE 2
Formula for Eudragit Enteric Coat Suspension.

| Ingredients | (g) |
|---|---|
| Eudragit L30D (30% solid Dispersion) | 368.6 |
| Triethyl Citrate | 16.5 |
| Talc | 22.1 |
| Anti-foam M | 1.1 |
| Purified Water | 327.9 |
| Total | 736.2 |

TABLE 3
Formula for subcoat for Opadry Enteric Coating Suspension

| Ingredients | Weight (g) |
|---|---|
| Opadry OY-S-7300G | 450 |
| Distilled Water | 2550 |
| Total | 3000 |
| | (15% Solid) |

TABLE 4
Formula for Opadry Enteric Coated Suspension (Top coat)

| Ingredients | Weight (g) |
|---|---|
| Opadry OY-A-7308* | 1620 |
| Distilled Water | 9180 |
| Ammonia Solution | 33 |
| Total | 10833 |
| | (15% solid) |

*available from Colorcon Ltd. Cray Orpington, Kent, England, and contains a specially modified polyvinyl acetate phthalate (74.5%), pigment (11.8%), plasticiser (11.2%) and other constituents (2.5%).

Method of Manufacture for Tablets

The manufacture of core tablets involves the use of conventional pharmaceutical equipment and processes. The process involves several stages including, sieving, blending, granulation or densification of materials (by Roller Compaction or slugging) to form a compression mix. Tablets are manufactured on a tabletting press using the appropriate size and shape punches. Finally, tablets are coated with an enteric coat using conventional coating equipment. During tablet coating, temperature and relative humidity of the system are controlled. A suitable process is described in WO 95/28927 (SmithKline Beecham).

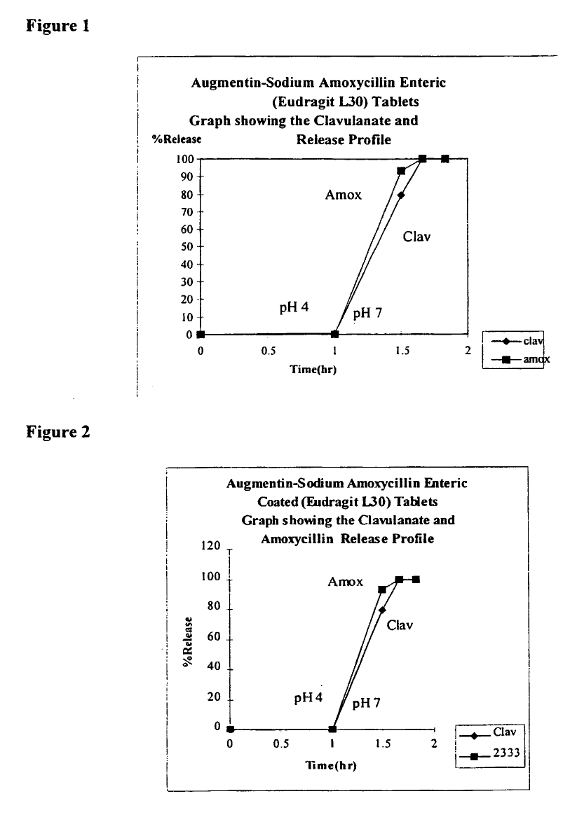

Dissolution tests were performed on the coated tablets described in Table 2 and 4 in pH 4 and pH 7 buffers. This demonstrated that the both enteric coating allows release of amoxycillin and clavulanic acid at a pH greater than pH 4. Results are shown in Tables 5 and 6 and illustrated in FIGS. 1 and 2.

TABLE 5
Release of actives from a Eudragit L30D Enteric Coated Tablet

| | Clavulanic | |
|---|---|---|
| | acid | Amoxycillin |
| Time (hr) | % Release | % Release |
| 0 | 0 | 0 |
| 1 | 0.3 | <0.1 |
| 1.5 | 79 | 93 |
| 1.7 | 100 | 100 |
| 1.8 | 100 | 100 |

TABLE 6
Showing the Release of Actives from an Opadry-A-7308 E Enteric
Coated Tablet

| | Clavulanic | |
|---|---|---|
| | acid | Amoxycillin |
| Time (hr) | % Release | % Release |
| 0 | 0 | 0 |
| 1 | 0.1 | <0.1 |
| 1.5 | 45 | 50 |
| 1.7 | 94 | 95 |
| 1.8 | 100 | 100 |

1. A tablet formulation comprising amoxycillin and potassium clavulanate, in a weight ratio amoxycillin: clavulanate between 1:1 to 20:1 (expressed as the weight of the corresponding parent acids) inclusive, wherein the amoxycillin is sodium amoxycillin or a mixture of sodium amoxycillin and amoxycillin trihydrate and the tablet has an enteric film coating.

2. A tablet formulation as claimed in claim 1 which comprises sodium amoxycillin and amoxycillin trihydrate in ratio between 2:1 and 10:1, preferably 3:1 to 5:1,, more preferably about 4:1 (expressed as the weight of the corresponding parent acid).

3. A tablet formulation as claimed in claim 1 or 2 in which the ratio of amoxycillin: clavulanate is selected from 2:1, 4:1, 7:12, 8:1, 14:1 and 16:1.

4. A tablet formulation according to any one of claims 1 to 3 in which the enteric coating is an enteric polymer.

5. A tablet formulation according to claim 4 in which the enteric polymer is selected from cellulose ethyl phthalate, polyvinylacetate phthalate, polyvinylbutyrate acetate, vinyl acetate-maleic anhydride copolymer, styrene-maleic mono-ester copolymer, methacrylic acid copolymers or methacrylate-methacrylic acid-octyl acrylate copolymer.

6. A tablet formulation according to claim 5 in which the enteric polymer is a methacrylic acid copolymer which is anionic in character and based on methacrylic acid and methyl or ethyl methacrylate having a ratio of free carboxyl groups: esterified carboxyl groups of 1:>3 and with a mean molecular weight greater than 100,000.

7. A tablet formulation according to claim 6 in which the ratio of free carboxyl groups to the ester is approximately 1:1 or 1:2.

8. A tablet formulation according to claim 7 in which the enteric film coating is a poly(methacrylic acid, ethyl acrylate) 1:1.

9. A tablet formulation according to any one of claims 1 to 8 in which the enteric film coating further comprises a plasticiser.

10. A tablet formulation according to any one of claims 1 to 9 in which the enteric coating further comprises an anti-tack agent.

11. A tablet formulation according to any one of the preceding claims comprising nominally 125/62.5, 250/62.5, 250/125, 500/62.5, 500/125, 875/125, 1000/62.5 and 1000/125 mg amoxycillin/clavulanate.

12. A method of treatment of bacterial infections in human beings or in animals comprising the oral administration to a human being or animal in need of such treatment of a medicament according to any one of claims 1 to 11.

13. A method for the preparation of a tablet formulation according to any one of claims 1 to 11 which method comprises coating the a tablet core with an enteric film coating.

14. A process as claimed in claim 13 in which the film coating is applied from an aqueous solution.

15. A tablet formulation comprising amoxycillin wherein the amoxycillin is sodium amoxycillin and optionally amoxycillin trihydrate and the tablet has an enteric film coating.
